(19) United States
(12) Patent Application Publication
(10) Pub. No.: US 2006/0058532 A1
(43) Pub. Date: Mar. 16, 2006

(54) Process for the scalable synthesis of 1,3,4,9-tetrahydropyrano[3,4-b]-indole derivatives

(76) Inventors: Warren Chew, Outremont (CA); Gloria Karen Cheal, Beaconsfield (CA); Jacqueline Francesca Lunetta, Pierrefonds (CA); Christopher A. Demerson, Kirkland (CA)

Correspondence Address: FITZPATRICK CELLA (WYETH), 30 ROCKEFELLER PLAZA, NEW YORK, NY, 10112-3800, US

(21) Appl. No.: 11/223,312

(22) Filed: Sep. 9, 2005

Related U.S. Application Data
(60) Provisional application No. 60/608,995, filed on Sep. 10, 2004.

(51) Int. Cl. C07D 209/36

(52) U.S. Cl. 548/485

(57) ABSTRACT

The invention is directed to a process of synthesizing compounds of formula (VI): 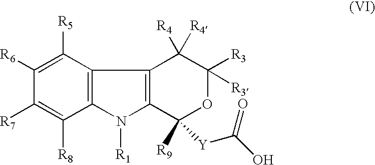
wherein R₁₋R₉, R_{3′}, R_{4′} and Y are as set forth in the specification, and said method is useful for large scale synthesis thereof. The invention is also directed to useful intermediates for synthesizing the compounds of formula (VI) and processes of preparing said intermediates.

### BACKGROUND OF THE INVENTION

This application claims the benefit of U.S. Provisional Application No. 60/608,995, filed Sep. 10, 2004.

### FIELD OF THE INVENTION

This invention is directed to a scalable process for synthesizing 1,3,4,9-tetrahydropyran[3,4-b]-indole derivatives and intermediates thereof.

### RELATED BACKGROUND ART

Pyranoindole derivatives have been shown to have activity that may be useful in the treatment of numerous disorders, including Hepatitis C, colorectal cancer, Alzheimer's disease, arthritis and other disorders associated with inflammation.

In the following U.S. patents, pyranoindole derivatives are disclosed and the compounds are stated to have antidepressant and antiulcer activity: U.S. Pat. Nos. 3,880,853 and 4,118,394. In U.S. Pat. No. 4,179,503 pyranoindoles are disclosed and stated to have diuretic activity. In the following U.S. patents, pyranoindole derivatives are disclosed and the compounds are stated to have antiinflammatory, analgesic, antibacterial, and antifungal activity: U.S. Pat. No. 3,843,681, 3,939,178, 3,974,179, 4,070,371, and 4,076,831. In the following U.S. patents, pyranoindole derivatives are disclosed and the compounds are stated to have antiinflammatory and analgesic activity: U.S. Pat. Nos. 4,670,462, 4,686,213, 4,785,015, 4,810,699, 4,822,781, and 4,960,902. In U.S. Pat. No. 5,776,967 and U.S. Pat. No. 5,830,911, pyranoindole derivatives are disclosed and the compounds are said to inhibit cyclooxegenase-2 and be useful for treating arthritic disorders, colorectal cancer, and Alzheimer's disease.

Also, in the following U.S. patents, processes for preparing pyranoindole derivatives are disclosed: U.S. Pat. Nos. 4,012,417, 4,036,842, 4,585,877, and 4,822,893. Processes for the resolution of racemic pyranoindole derivatives are disclosed in U.S. Pat. Nos. 4,501,899, 4,515,961, 4,520,203, and 4,544,757.

In U.S. Pat. No. 4,822,893, a process for synthesizing pyranoindole derivatives from a tryptophol intermediate is described, wherein the intermediate is formed either by condensing a phenylhydrazine with a 2,3-dihydrofuran, with the subsequent cyclization occurring under acidic conditions, or alkylating an isatin with ethyl or methyl propionate. Similarly, U.S. Pat. No. 4,012,417 discloses forming the tryptophol intermediate by reacting a phenylhydrazine with a hydroxyaldehyde. These processes, however, require that the intermediate be purified before being reacted in subsequent steps. Therefore, there is need for a process of synthesizing pyranoindole derivatives from a tryptophol intermediate wherein the intermediate is obtained sufficiently pure so that it may be used in a subsequent step without chromatographic purification. A process such as this would be ideal for large scale preparative synthesis of pyranoindole derivatives, because large scale purifications can be difficult to perform, and in the case of chromatographic purification just about impossible.

### BRIEF SUMMARY OF THE INVENTION

This invention is directed to a process of synthesizing compounds of formula (VI): 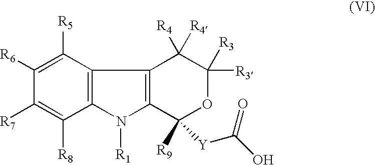
from compounds of formula (V) 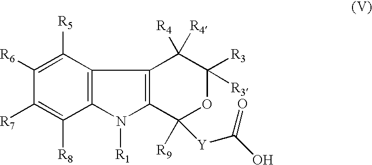
   wherein R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms, all of which may be optionally substituted; R₃ and R_{3′} are H; R₄ and R_{4′} are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an aryl of 6 to 12 carbon atoms, a furanylmethyl, an arylalkyl or alkylaryl of 7 to 12 carbon atoms, an alkynyl of 2 to 7 carbon atoms, all of which may be optionally substituted, or R₄ and R_{4′} taken together with the ring carbon atom to which they are attached are a carbonyl group; R₅-R₈ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, F, Cl, Br, I, CN, CF₃, NO₂, an alkylsulfinyl of 1 to 8 carbon atoms, an alkylsulfonyl of 1 to 6 carbon atoms, a pyrrolidinyl, or a thiazolidinyl, all of which can be optionally substituted; R₁₂ and R₁₃ are independently H, a straight chain alkyl of 1 to 8 carbon atoms, a branched alkyl of 3 to 12 carbon atoms, a cycloalkyl of 3 to 12 carbon atoms, an aryl of 6 to 12 carbon atoms or a heterocycloalkyl of 2 to 9 carbon atoms, all of which can be optionally substituted; R₉ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all of which can be optionally substituted; and Y is a bond, CH₂, CH₂CH₂, aryl of 6 to 12 carbon atoms, or R₉ and Y together with the ring carbon atom to which they are attached may additionally form a spirocyclic cycloalkyl ring of 3 to 8 carbon atoms; and said process comprises the step of dissolving the compound of formula (V) with a resolving agent to obtain the compound of formula (VI) by recrystallization.

The present invention also relates to a process of synthesizing compounds of formula (I): 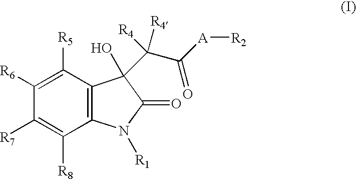
comprising the steps of reacting a compound of formula (II) 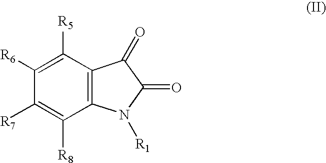
with a reagent of formula M^{+-C(R}₄R_{4′})C(O)-A-R₂, wherein:
R₂ is a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all which may be optionally substituted or unsubstituted; A is O or S; and R₁, R₄-R₈, R_{4′} and A are as defined above and M⁺ is a metal cation.

Another aspect of the present invention are compounds of formula (I): 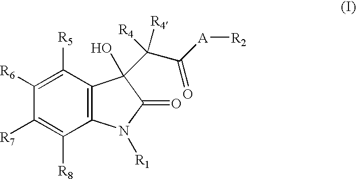
which are useful intermediates in the synthesis of compounds of formulas (V) and (VI): 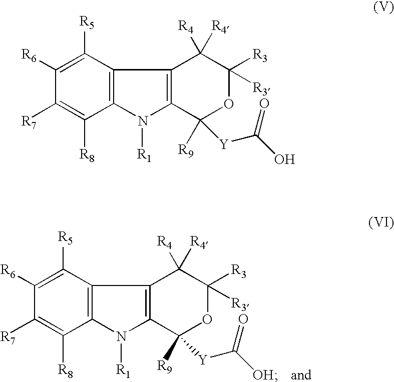
R₁-R₉, R_{3′}, R_{4′} and A are as defined above.

### DETAILED DESCRIPTION

In the present invention compounds of formula (VI) are synthesized from compounds of formula (I) without the need of chromatography. The only purification necessary in this process is a recrystallization to effect an enantiomeric resolution of the final product.

Using a common reducing agent, a compound of formula (I) is reduced to the corresponding tryptophol of formula (III). This tryptophol compound is then reacted with a reagent of formula R₉—C(O)—Y—CO₂R₁, wherein R₉, Y and R₁₁ are as defined herein, under acidic conditions to obtain a pyranoindole ester of formula (IV). The pyranoindole ester is then hydrolyzed to the corresponding acid of formula (V). The enantiomerically pure final product of formula (VI) is then obtained by recrystallizing the pyranoindole acid of formula (V) with a resolving agent. As this process allows for a multi-step synthesis of the product without the need for purification until the enantiomeric resolution, it is ideal for use for large-scale preparation of compounds of formula (VI).

Another aspect of this invention is the process of preparing the compounds of formula (I), which are the starting materials used in the above-described method. An aniline of formula (VII) is first reacted with a trihaloacetaldehyde hydrate and hydroxylamine hydrochloride to form a compound of formula (VIII), which is subsequently cyclized in the presence of an acid to give the corresponding isatin of formula (II). This isatin is then reacted with an organo-metalic reagent of formula M⁺⁻C(R₄R_{4′})C(O)-A-R₂, wherein M⁺ is a metal cation and A, R₂, R₄ and R_{4′} are as defined herein, to obtain the corresponding compound of formula (I). This process for preparing the compounds of formula (I) also does not require any purification and furthermore, the compounds of formula (I) can be used to synthesize the compounds of formula (VI), as detailed above, without any purification. Thus, using the methodologies described herein, a final product of formula (VI) can be synthesized from the starting aniline of formula (VII) without any purification until the enantiomeric resolution performed in the last step.

For purposes of this invention the term “alkyl” includes straight chain moieties with a length of up to 12 carbon atoms, but preferably 1 to 8 carbon atoms, and more preferably 1 to 4 carbons. The term “alkyl” also includes branched moieties of 3 to 12 carbon atoms. The term “alkenyl” refers to a radical aliphatic hydrocarbon containing one double bond and includes both straight and branched alkenyl moieties of 2 to 7 carbon atoms. Such alkenyl moieties may exist in the E or Z configurations; the compounds of this invention include both configurations. The term “alkynyl” includes both straight chain and branched moieties containing 2 to 7 carbon atoms having at least one triple bond. The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl.

For purposes of this invention the term “aryl” is defined as an aromatic hydrocarbon moiety and may be substituted or unsubstituted. An aryl may be selected from but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl. In one embodiment the substituted aryl may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂. Preferred substituents for aryl and heterocycloalkyl include: alkyl, halogen, amino, alkylamino, dialkylamino, trifluoromethyl, trifluoromethoxy, arylalkyl, and alkylaryl. Preferably an aryl group consists of 6 to 12 carbon atoms.

For purposes of this invention the term “heterocycloalkyl” is defined as 5-14 membered aromatic, partially saturated or saturated heterocyclic ring system (monocyclic or bicyclic or tricyclic) where the heterocyclic moieties are five or six membered rings containing 1 to 4 heteroatoms selected from the group consisting of S, N, and O, and include but are not limited to: (1) five or six membered rings such as furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-thazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having one nitrogen atom, such as quinoline; (ii) fused to a 5 or 6-membered aromatic (unsaturated) heterocyclic ring having two nitrogen atoms such as quinazoline; (iii) fused to a 5-membered aromatic (unsaturated) heterocyclic ring having one nitrogen atom together with either one oxygen or one sulfur atom such as benzoxazole, benzothiazole, benzisoxazole, benzimidazole, N-methylbenzimidazole, azabenzimidazole, indazole; or (iv) fused to a 5-membered aromatic (unsaturated) heterocyclic ring having one heteroatom selected from O, N or S such as indole, benzofuran, azaindole. Preferably a heterocycloaklyl group consists of 2 to 9 carbon atoms. Saturated or partially saturated heterocycloalkyl groups include heterocyclic rings selected from but not limited to the moieties: azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

For the purposes of this invention the term “alkoxy” is defined as C₁-C₁₂-alkyl-O—, but preferably consists of 1 to 8 carbon atoms; the term “aryloxy” is defined as aryl-O—; the term “heterocycloalkyloxy” is defined as heterocycloalkyl-O—; wherein alkyl, aryl, and heterocycloalkyl are as defined above.

For purposes of this invention the term “arylalkyl” is defined as aryl-C₁-C₆-alkyl-, but preferably the entire moiety contains 7 to 12 carbon atoms. Arylalkyl moieties include benzyl, 1-phenylethyl, 2-phenylethyl, 3-phenylpropyl, 2-phenylpropyl and the like.

For purposes of this invention the term “alkylaryl” is defined as C₁-C₆-alkyl-aryl-, but preferably the entire moiety contains 7 to 12 carbon atoms.

For purposes of this invention the term “alkylthio” is defined as C₁-C₆-alkyl-S—.

For purposes of this invention “alkoxyalkyl,” “cycloalkyl-alkyl,” and “alkylthioalkyl,” denotes an alkyl group as defined above that is further substituted with an alkoxy, cycloalkyl or alkylthio group as defined above. Preferably, a “cycloalkyl-alkyl” moiety consisting of 4 to 24 carbon atoms, and a “alkylthioalkyl” moiety consists of C₁-C₆-alkyl-S—C₁-C₁₂-alkyl-, but preferably consists of 2 to 16 carbon atoms.

For purposes of this invention “arylalkoxy,” and “fluoroalkoxy,” denote an alkoxy group as defined above that is further substituted with an aryl group, as defined above, or at least one fluoro atom. Preferably, an “arylalkoxy” moiety consists of 7 to 12 carbon atoms.

For purposes of this invention “phenylalkynyl” is an alkynyl group further substituted with a phenyl group.

The terms “monoalkylamino” and “dialkylamino” refer to moieties with one or two alkyl groups wherein the alkyl chain is 1 to 8 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1 to 8 carbon atoms.

“Acyl” is a radical of the formula —(C═O)-alkyl or —(C═O)-perfluoroalkyl wherein the alkyl radical or perfluoroalkyl radical is 1 to 7 carbon atoms; preferred examples include but are not limited to, acetyl, propionyl, butyryl, trifluoroacetyl.

For purposes of this invention the term “alkylsulfinyl” is defined as a R′SO— radical, where R′ is an alkyl radical of 1 to 8 carbon atoms. Alkylsulfonyl is a R′SO₂— radical, where R′ is an alkyl radical of 1 to 6 carbon atoms. Alkylsulfonamido, alkenylsulfonamido, alkynylsulfonamido are R′SO₂NH— radicals, where R′ is an alkyl radical of 1 to 8 carbon atoms, an alkenyl radical of 2 to 8 carbon atoms, or an alkynyl radical of 2 to 8 carbon atoms, respectively.

The term “cyanoalkyl” refers to an alkyl radical, as defined above, that is further substituted with a cyano group. The preferred embodiment is wherein the alkyl radical contains 1 to 8 carbon atoms.

The terms “carbonyl” and “oxo” refer to a —C(O)— moiety.

For the purpose of this invention the term “trihaloacetaldehyde hydrate” refers to a compound of formula CX₃CH(OH)₂, wherein X is a halogen. One example of such a compound is chloral hydrate.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halo, nitro, amino, hydroxyl, cyano, alkylamino, dialkylamino, alkoxy, haloalkoxy, alkylthio, mercapto, haloalkylthio, aryl, aryloxy, arylthio, heterocycloalkyl, heterocycloalkyloxy, heterocycloalkylthio or acyl. This list is provided for illustrative purposes and is not intended to be exhaustive.

For the purposes of this invention the term “substituted” refers to where a hydrogen radical on a molecule has been replaced by another atom radical, a functional group radical or a moiety radical; these radicals being generally referred to as “substituents.”

The compounds prepared by the process of this invention may contain an asymmetric carbon atom and some of the compounds of this invention may contain one or more asymmetric centers and may thus give rise to stereoisomers, such as enantiomers and diastereomers. The stereoisomers of the instant invention are named according to the Cahn-Ingold-Prelog System. While shown without respect to stereochemistry in Formulas (I) and (V), the present invention includes all the individual possible stereoisomers; as well as the racemic mixtures and other mixtures of R and S stereoisomers (scalemic mixtures which are mixtures of unequal amounts of enantiomers) unless otherwise specified, such as in Formula (VI). It should be noted that stereoisomers of the invention having the same relative configuration at a chiral center may nevertheless have different R and S designations depending on the substitution at the indicated chiral center.

For compounds described herein containing two chiral centers, four possible stereoisomers are possible; these four stereoisomers are classified as two racemic pairs of diastereomers. These compounds may be present as racemic diastereomers which would be designated following the convention described in the 1997 Chemical Abstracts Index Guide, Appendix IV (Columbus, Ohio) whereas the first cited chiral atom is designated R* and the next cited chiral atom is designated R* if it possesses the same chirality as the first cited stereocenter or S* if it possesses opposite chirality to the first cited stereocenter. Alternatively, these compounds of the invention may be present as non-racemic mixtures of two diastereomers owing to the existence of a predefined stereocenter. In these instances, the predefined stereocenter is assigned based on the Cahn-Ingold-Prelog System and the undefined stereocenter is designated R* to denote a mixture of both R and S stereoisomers at this center. Compounds of this invention which possess two chiral centers but which are present as single stereoisomers are described using the Cahn-Ingold-Prelog System.

One possible embodiment of the compounds of formula (I) is wherein R₁ is H or C₁-C₄ alkyl; R₂ is a group selected from C₁-C₈ alkyl, C₇-C₁₂ alkyl-aryl, aryl and heterocycloalkyl, more preferably C₁-C₄ alkyl or aryl; R₃, R_{3′}, R₄ and R_{4′} are H; R₅-R₈ are independently H, C₁-C₄ alkyl, F, Cl, Br, CN or CF₃; and A is preferably 0.

A specific embodiment of the compounds of formula (I) is wherein R₁, R₃, R_{3′}, R₄ and R_{4′}, R₆ and R₇ are H, R₂ is t-butyl, R₅ is Br and R₈ is CH₃.

In one embodiment of the process of this invention the tryptophol intermediate is synthesized using a modified Sandmeyer methodology., T. Sandmeyer, Helv. Chem. Acta. Vol. 2, pp. 234 (1919), which is hereby incorporated by reference. This methodology provides the benefit of obtaining the intermediate in sufficient purity and thus, it may be used in a subsequent step without further purification. This is a major improvement over the prior methods, which required that the intermediate be chromatographically purified. The process of synthesis of the present invention requires no chromatographic purification from start to finish. For this reason, the process is ideal for large-scale preparative synthesis of pyranoindole derivatives.

Various embodiments of the process of the present invention are represented by Schemes I and II below:

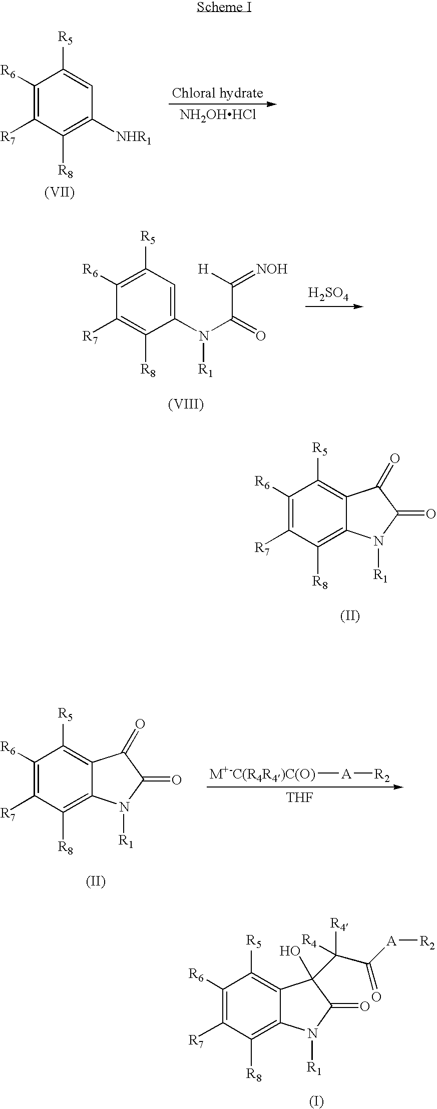

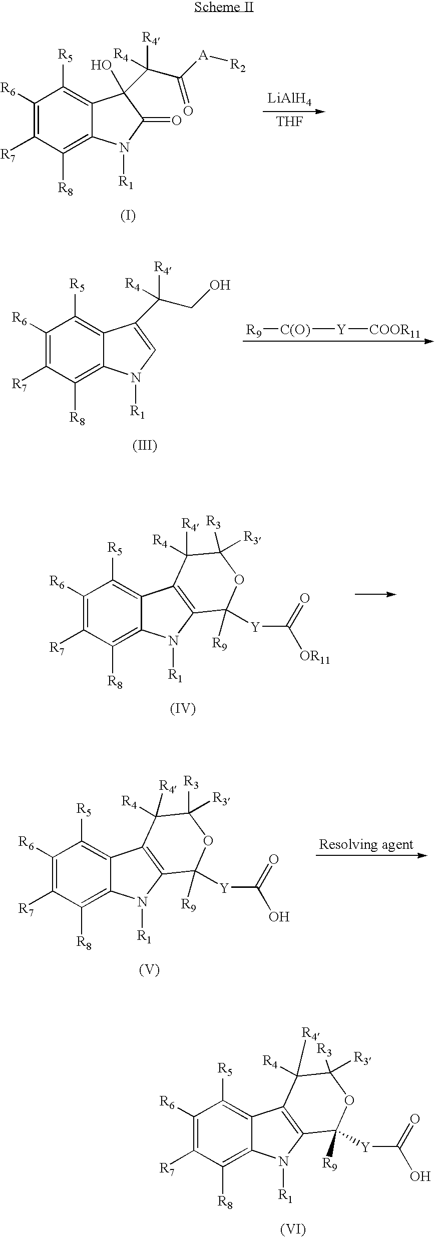

In Scheme I, a compound of formula (VII), wherein R₁ and R₅-R₈ are as defined supra, is reacted with a trihaloacetaldehyde hydrate, such as chloral hydrate, and hydroxylamine hydrochloride to produce a compound of formula (VIII).

The compound of formula (VIII) is then cyclized in the presence of an acid to give a corresponding isatin, as defined by formula (II). The acid can be a strong mineral acid or a Lewis acid. Preferably the acid is sulfuric acid.

To form a compound of formula (I), the isatin of formula (II) is reacted with an organo-metalic reagent of the formula M⁺⁻C(R₄R_{4′})C(O)-A-R₂, wherein M⁺ is a metal cation, A is an oxygen or a sulfur atom, and R₂, R₄ and R_{4′} are as defined supra. Exemplary metal cations include Na⁺, K⁺, and Li⁺. One skilled in the art can readily generate the organo-metalic reagent, for example by reacting the corresponding organic compound with a metal hydride, such as NaH or KH, or a strong organo-metalic-base, such as LiN(TMS)₂, n-butyl Li or t-butyl Li. In one embodiment the organo-metalic reagent is formed by reacting LiN(TMS)₂ with t-butyl acetate.

In another embodiment of the process shown in Scheme I, the compounds used or formed are defined such that R₁ is H or C₁-C₄ alkyl; R₂ is a group selected from C₁-C₈ alkyl, C₇-C₁₂ alkyl-aryl, aryl and heterocycloalkyl, but in a more specific embodiment R₂ is a C₁-C₄ alkyl or aryl group; R₃, R₃′, R₄ and R₄ are H; R₅-R₈ are independently H, C₁-C₄ alkyl, F, Cl, Br, CN or CF₃; and A is O or S.

In a specific embodiment of the process shown in scheme I, the compounds used or formed are defined such that R₁, R₃, R_{3′}, R₄, R_{4′}, R₆ and R₇ are H; R₂ is t-butyl; R₅ is Br; and R₈ is methyl.

Another embodiment of the process shown in Scheme I is where the entire synthesis of the compound of formula (I) is performed without any chromatographic purifications.

Scheme II illustrates that a stereo-specific pyranoindole derivative of formula (VI) can be synthesized from the compound of formula (I).

The compound of formula (I) is first reduced to the corresponding tryptophol, defined by formula (III). This reduction can be effected with reducing reagents such as LiAlH₄ or NaBH₄ and BF₃.Et₂O. Other reducing agents are possible and one skilled in the art would be aware of these reagents. This reduction provides the tryptophol compound in sufficient purity. Therefore, no chromatography, or any other purification, is necessary in order to take the compound forward into the next step of the synthesis.

The tryptophol of formula (III) is then reacted with a reagent of the formula R₉—C(O)—Y—CO₂R₁₁, wherein R₉ and Y are as defined supra and R₁₁ includes groups selected from alkyl, alkenyl, alkynyl, alkoxyalkyl, arylalkyl, alkylthioalkyl, cycloalkyl-alkyl, heterocycloalkyl, or aryl wherein any of these groups may be optionally substituted or unsubstituted. This reaction is done in the presence of an acid to give a compound of formula (IV). One skilled in the art would readily be able to determine suitable acids for use in this reaction. Lewis acids, such as BF₃.Et₂O, ZnCl₂, AlCl₃, BCl₃, BBr₃ and FeCl₃ work well. For this reaction exemplary solvents include THF, Et₂O and EtOAc, but one skilled in the art would know of other suitable solvents.

Hydrolysis of the pyranoindole ester of formula (IV) follows to give a compound of formula (V). This hydrolysis can be performed under acidic, basic or neutral conditions, depending on the nature of the R₁₁ group. One skilled in the art would understand this and know, based upon the R₁₁ group, which conditions would be appropriate.

The racemic pyranoindole acetic acid of formula (V) can then be recrystallized in the presence of a resolving agent to give the pure (R) enantiomer of a compound of formula (VI). This recrystallization can be done in a solvent such as methanol, ethanol or a similar alkyl alcohol. Additionally, a co-solvent may also be used. Typical co-solvents used with alcohols are, hexanes, ethyl ether, ethyl acetate, acetone and methyl ethyl ketone (MEK). One skilled in the art would be aware of numerous other solvents commonly employed in recrystallizations. The literature is repleat with the numerous resolving agents which could be employed in this recrystallization, such as (+) cinchonine, (−) burcine, (−) ephedrine, R-(−)-2-amino-1-butanol, R-(−)-2-amino-1-propanol, R-(−)-2-amino-3-methyl-1-butanol, R-(+)-2-amino-3-3-dimethylbutane, R-(+)-2-amino-3-phenyl-1-propanol, (R)-phenylethylamine, (S)-phenylethylamine, S-(+)-2-amino-1-butanol, S-(+)-2-amino-1-propanol, S-(+)-2-amino-3-methyl-1-butanol, N-methyl-D-glucamine, (R)-(+)-N,N-dimethyl-1-phenethylamine, (S)-(−)-N,N-dimethyl-1-phenethylamine, (1R,2R)-(−)-pseudoephedrine, (1R,2S)-(−)-ephedrine, (1 S,2S)-(+)-pseudoephedrine, (R)-(−)-ephinephrine, nicotine, quinine, strychnine and the like. One skilled in the art would be aware of other similar reagents. (+) Cinchonine is preferred.

The salt crystals recovered from the recrystallization are then dissolved in a mixture of a suitably water-immiscible organic solvent, such as toluene, EtOAc, CH₂Cl₂ or the like, and an aqueous acid solution, such as 1 to 6 normal HCl, H₂SO₄ or the like. The organic solvent is then isolated and removed to give the enantiomeric pure compound of formula (VI).

In an embodiment of the process shown in Scheme II, the compounds reacted or formed are defined such that R₁ is H or C₁-C₄ alkyl; R₂ is a group selected from C₁-C₈ alkyl, C₇-C₁₂ alkylaryl, C₆-C₁₂ aryl and C₆-C₉ heterocycloalkyl; R₃, R_{3′}, R₄ and R_{4′} are H; R₅-R₈ are independently H, C₁-C₄ alkyl, F, Cl, Br, CN or CF₃; A is O or S; R₉ is H or C₁-C₈ alkyl; and Y is a bond, CH₂, CH₂CH₂, or C₆-C₁₂ aryl, or R₉ and Y together with the ring carbon atom to which they are attached may additionally form a spirocyclic cycloalkyl ring of 3 to 8 carbon atoms. A more specific embodiment is where R₂ is C₁-C₄ alkyl or aryl, R₉ is H or C₁-C₄ alkyl, and Y is CH₂.

In another embodiment of the process shown in scheme II, is wherein the compounds reacted or formed are defined by R₁ being H, R₅-R₈ being independently selected from H, a straight chain alkyl of 1 to 4 carbons, F, Br, Cl or CN, A is O, and R₉ being H or a straight chain alkyl of 1 to 4 carbons. A specific embodiment of this is wherein R₂ is t-butyl, R₅ is CN, R₆ and R₇ are H, R₈ is CH₃, and R₉ is n-propyl.

Compounds of formulas (I) and/or (IV), wherein at least one of R₅-R₈ is a leaving group selected from the group consisting of halo, —O-triflate, —O-mesylate, or —O-tosylate, can be further derivatized by arylation prior to reacting them in their respective next steps, as shown in Scheme II. The arylation can occur under non-acidic conditions using a variety of reagents. Compounds with aryl leaving groups, such as those disclosed above, can be converted into arylcyanides, arylalkanes, biaryls, arylalkynes and aryl alkane ethers. This is not meant to be an exhaustive list and one skilled in the art would know of other possible products.

Another embodiment of the process shown in Scheme II is where the entire synthesis of the compound of formula (VI), including the possible arylation step discussed above, is performed without any chromatographic purifications.

The specific synthesis of (R) 5-cyano-8-methyl-1-propyl-1,3,4,9-tetrahydropyran[3,4b]-indolyl-1-acetic acid, example 1, is illustrated below in Scheme III.

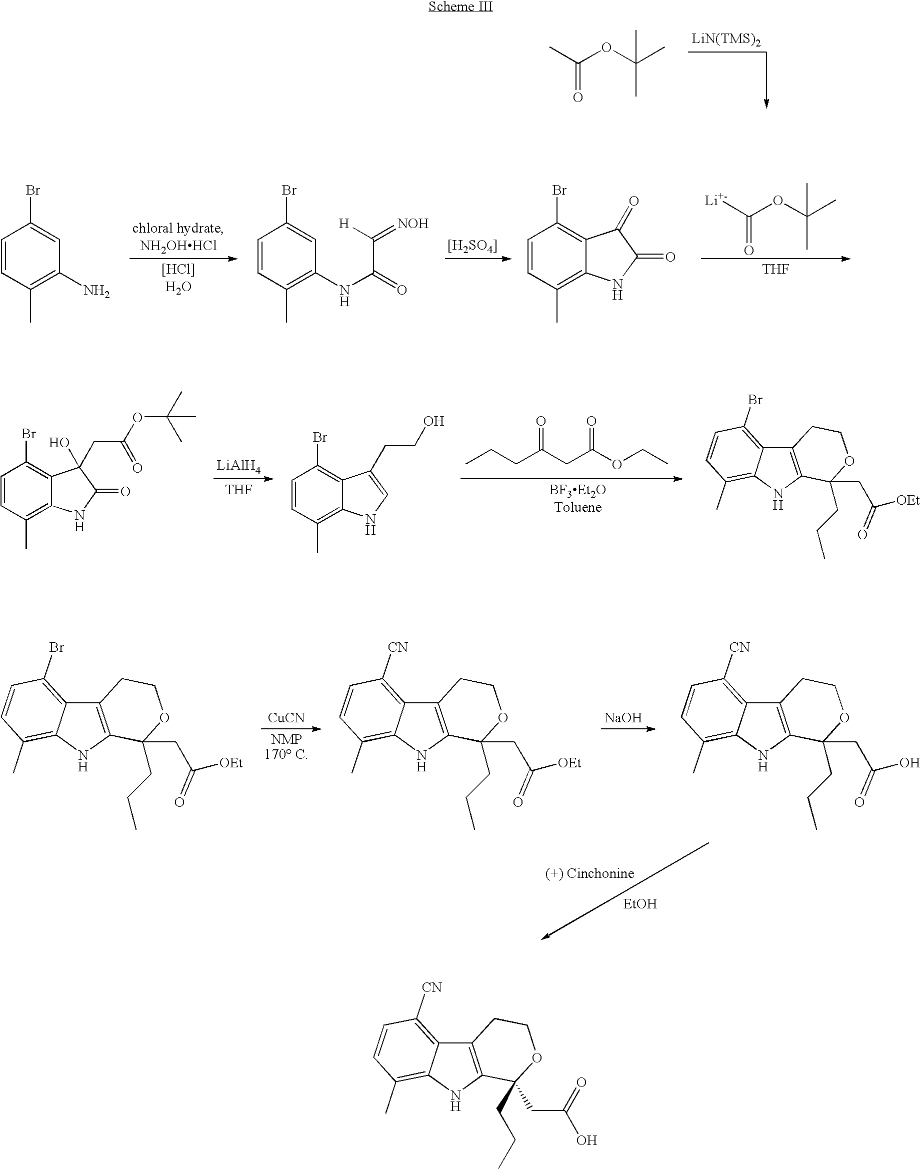

### Preparation of 4-Bromo-7-methylisatin

To a mixture of chloral hydrate (0.39 kg, 2.36 mole) in water (3.6 L) was charged sodium sulfate (1.22 kg). A mixture of 5-bromo-2-methylaniline (0.40 kg, 2.15 mole), water (1.84 L) and concentrated HCl (0.22 kg) were added to the aqueous chloral hydrate mixture followed by a solution of hydroxylamine hydrochloride (0.488 kg) in water (0.96 L). The mixture was heated to 70-75° C. and stirred for a minimum of 6 h until less than ˜10% 5-bromo-2-methylaniline remains by TLC. The mixture was cooled to room temperature, filtered and the cake washed with water (2×1.2 L). The wet solid (5-bromo-2-methylisonitrosoacetanilide) was added to hot sulfuric acid (2.94 kg) at 70-75° C. and stirred for a minimum of 30 mins until less than ˜2% starting material remains by TLC. The mixture was cooled and quenched into ice water (6.4 L) over 40 mins. The precipitated solids are filtered, reslurried in water (2.4 L) and filtered. The wet cake was washed with heptane (3×0.80 L). The solid was dried (65° C., 10 mm Hg, 24-48 h) to give 4-bromo-7-methyl isatin in 63% overall yield from the starting aniline.

### Preparation of t-Butyl 4-bromo-2,3-dihydro-3-hydroxy-7-methyl-2-oxo-1H-indolyl-3-acetate

A stirred mixture of t-butyl acetate (0.725 kg) in THF (1.45 L) was cooled to −45±5° C. A 1 M THF solution of lithium bis(trimethylsilyl)amide (6.24 L) was added while maintaining the temperature between −45±5° C. After 30 min, a slurry of 4-bromo-7-methyl isatin (0.30 kg) in THF (1.50 L) was added to the solution and the mixture allowed to warm to room temperature over 30 mins. The reaction was complete when less than 5% of the isatin remains by TLC. The mixture was concentrated to a volume of 3.5 L and cooled to 0-10° C. The mixture was quenched with water (0.67 L) and acidified to pH 2-3 with 6 N HCl (˜2.1 L). The mixture was extracted with ethyl acetate (2×2.33 L), washed with water (3.2 L), 10% brine (2.67 L) and dried over sodium sulfate (0.67 kg). The organic solvents were concentrated to a volume of ˜0.90 L to precipitate the product. Heptane (0.67 L) was added to further precipitate the product. The mixture was cooled and the solid was filtered and washed with heptane (2×0.33 L). The solid was dried (65° C., 10 mm Hg, 24-48 h) to give the product in 50% yield.

### Preparation of 4-Bromo-7-methyl tryptophol

A stirred mixture of t-butyl 4-bromo-2,3-dihydro-3-hydroxy-7-methyl-2-oxo-1H-indolyl-3-acetate (0.215 kg) in THF (1.08 L) was cooled to 0-10° C. A 1 M THF solution of lithium aluminum hydride (1.75 L) was added over 1.5-2 h maintaining 0-10° C. The mixture was held for 30 mins, heated to reflux for 2.5 h then cooled to room temperature. The reaction was complete when less than 1% of the starting material remains by TLC. The reaction was further cooled to 0-10° C. and quenched with ethyl acetate (1.0 L) and water (0.063 L) and then acidified to pH 2-3 with 6N HCl (˜1.6 L). The organic layer was separated and the aqueous layer was extracted with ethyl acetate (0.32 L). The combined organic layers were washed sequentially with water (1.0 L) and 10% brine (1.0 L) and then dried over sodium sulfate (0.32 kg). The solution was distilled to an oil to give crude tryptophol which was used without further purification.

### Preparation of Ethyl 5-bromo-8-methyl-1-propyl-1,3,4,9-tetrahydropyrano[3,4b]-indolyl-1-acetate

Crude tryptophol (0.107 kg) was dissolved in toluene (1.81 L). The solution was cooled to 10-15° C. and ethyl butyryl acetate (0.067 kg) was added followed by boron trifluoride diethyl etherate (0.060 kg). The mixture was stirred for a minimum of 2 h until less than 1% tryptophol remains by HPLC. The reaction was quenched with a solution of sodium bicarbonate (0.022 kg) in water (0.27 L) and filtered to remove insolubles. The filtrates were separated and the organic layer washed sequentially with 8% aqueous sodium bicarbonate (0.27 L), 10% brine (2×0.21 L), water (0.21 L) and 10% brine (0.21 L). The organic layer was then dried over sodium sulfate (0.15 kg). The solution was distilled to an oil (˜0.18 L) to give the pyranoindole which was used without further purification.

### Preparation of Ethyl 5-cyan-8-methyl-1-propyl-1,3,4,9-tetrahydropyrano[3,4b]-indolyl-1-acetate

Crude pyranoindole (130-140 g) was dissolved in NMP (1.9 L) and the solution distilled to remove residual toluene. Copper cyanide (0.060 kg) was added and the mixture was heated to 170° C. for 5 h until less than 1% bromo pyranoindole remains by HPLC. The mixture was cooled to room temperature and quenched into water (10.0 L). Ethyl acetate (4.0 L) was added and the mixture filtered over celite and washed with a mixture of water (0.20 L) and ethyl acetate (0.10 L). The organic layer was separated and the aqueous backwashed with ethyl acetate (3.0 L). The combined organic layers were washed with 10% brine (2×0.75 L), water (0.75 L) and dried over sodium sulfate (0.15 kg). The solution was distilled to semi-solid that was purified by slurrying in ethanol (0.23 L). The mixture was filtered and washed with ethanol (0.065 L). The resulting solid was dried (40° C., 10 mm Hg, 24-48 h) to give the product as an off-white solid in 50% over 3 steps.

### Preparation of 5-Cyano-8-methyl-1-propel-1,3,4,9-tetrahydropyrano[3,4b]-indolyl-1-acetic acid

To a stirred mixture of ethyl 5-cyan-8-methyl-1-propyl-1,3,4,9-tetrahydro-pyrano[3,4b]-indolyl-1-acetate (0.068 kg) in 3:1 THF:water (1.36 L) was added 1 N NaOH (0.38 L) over 20 min at room temperature. The solution was stirred at room temperature until hydrolysis (<1% starting material) was complete by HPLC. THF was removed by distillation and the basic aqueous layer was extracted with heptane (2×0.20 L). The aqueous layer was cooled to 0-10° C. and acidified to pH 2-3 with 1N HCl (˜0.40 L). The resulting mixture was stirred for 30 mins, filtered and washed with cold water (0.14 L). The solid was dried (40° C., 10 mm Hg, 4-24 h) to give the product in 98% yield.

### (R) 5-Cyano-8-methyl-1-propyl-1,3,4,9-tetrahydropyrano[3,4b]-indolyl-1-acetic acid

A stirred mixture of racemic 5-cyan-8-methyl-1-propyl-1,3,4,9-tetrahydro-pyrano[3,4b]-indolyl-1-acetic acid (0.465 kg) and (+) cinchonine (0.531 kg) in ethanol (6.97 L) was heated at reflux (78-80° C.) for 2 h. The mixture was seeded with the cinchonine salt of the product (0.30 g) and progressively cooled to room temperature over 11 h. The resulting solid was filtered and washed with cold ethanol (3×0.25 L) to provide the (R)-cinchonine salt (0.30 kg) in greater than 85% enantiopurity. The salt was recrystallized a second time in ethanol to provide the salt in >99.5% enantiopurity. The solid was dried (45° C., 10 mm Hg, 2 h) to provide 0.28 kg. The salt was suspended in ethyl acetate (2.50 L). 1 N HCl (1.20 L) was added and the mixture was stirred at room temperature for 10 min. The clear layers were separated, and the aqueous layer backwashed with ethyl acetate (0.50 L). The combined organic layers were washed with 1 N HCl (0.50 L), water (1.0 L) and 10% brine (1.0 L) and dried over sodium sulfate (0.30 kg). The mixture was concentrated to a volume of ˜1.0 L and heptanes (4.50 L) was added to precipitate the product. The mixture was cooled to 0-5° C., filtered, washed with cold heptanes (2×0.25 L). The product was dried (55° C., 10 mm Hg, 24 h) to give the free acid (0.102 kg, 22% yield). Residual cinchonine in the product can be removed by additional 1 N HCl washes. The product may be recrystallized from IPA/water. The filtrate from the first drop of the cinchonine salt was predominantly the (S)-enantiomer, which can be racemized and recycled to provide additional (R)-enantiomer.

### EXAMPLE 1

### (R) 5-Cyano-8-methyl-1-propyl-1,3,4,9-tetrahydropyrano[3,4b]-indolyl-1-acetic acid

This compound was synthesized as discussed above and illustrated in Scheme III.

### EXAMPLE 2

### 4-Chloro-7-methylisatin

To a mixture of chloral hydrate (0.39 kg, 2.36 mole) in water (3.6 L) was charged sodium sulfate (1.22 kg). A mixture of 5-chloro-2-methylaniline (0.40 kg, 2.15 mole), water (1.84 L) and concentrated HCl (0.22 kg) were added to the aqueous chloral hydrate mixture followed by a solution of hydroxylamine hydrochloride (0.488 kg) in water (0.96 L). The mixture was heated to 70-75° C. and stirred for a minimum of 6 h until less than ˜10% 5-chloro-2-methylaniline remains by TLC. The mixture was cooled to room temperature, filtered and the cake washed with water (2×1.2 L). The wet solid (5-chloro-2-methylisonitrosoacetanilide) was added to hot sulfuric acid (2.94 kg) at 70-75° C. and stirred for a minimum of 30 mins until less than ˜2% starting material remains by TLC. The mixture was cooled and quenched into ice water (6.4 L) over 40 mins. The precipitated solids are filtered, reslurried in water (2.4 L) and filtered. The wet cake was washed with heptane (3×0.80 L). The solid was dried (65° C., 10 mm Hg, 24-48 h) to give 4-chloro-7-methyl isatin in 63% overall yield from the starting aniline.

### EXAMPLE 3

### t-Butyl 4-chloro-2,3-dihydro-3-hydroxy-7-methyl-2-oxo-1H-indolyl-3-acetate

A stirred mixture of t-butyl acetate (0.725 kg) in THF (1.45 L) was cooled to −45±5° C. A 1 M THF solution of lithium bis(trimethylsilyl)amide (6.24 L) was added while maintaining the temperature between −45±5° C. After 30 min, a slurry of 4-chloro-7-methyl isatin (0.30 kg) in THF (1.50 L) was added to the solution and the mixture allowed to warm to room temperature over 30 mins. The reaction was complete when less than 5% of the isatin remains by TLC. The mixture was concentrated to a volume of ˜3.5 L and cooled to 0-10° C. The mixture was quenched with water (0.67 L) and acidified to pH 2-3 with 6 N HCl (−2.1 L). The mixture was extracted with ethyl acetate (2×2.33 L), washed with water (3.2 L), 10% brine (2.67 L) and dried over sodium sulfate (0.67 kg). The organic solvents are concentrated to a volume of ˜0.90 L to precipitate the product. Heptane (0.67 L) was added to further precipitate the product. The mixture was cooled and the solid was filtered and washed with heptane (2×0.33 L). The solid was dried (65° C., 10 mm Hg, 24-48 h) to give the product in 50% yield.

### EXAMPLE 4

### Ethyl 4-bromo-2,3-dihydro-3-hydroxy-7-methyl-2-oxo-1H-indolyl-3-acetate

A stirred mixture of ethyl acetate (0.725 kg) in THF (1.45 L) was cooled to −45±5° C. A 1 M THF solution of lithium bis(trimethylsilyl)amide (6.24 L) was added while maintaining the temperature between −45±5° C. After 30 min, a slurry of 4-bromo-7-methyl isatin (0.30 kg) in THF (1.50 L) was added to the solution and the mixture allowed to warm to room temperature over 30 mins. The reaction was complete when less than 5% of the isatin remains by TLC. The mixture was concentrated to a volume of ˜3.5 L and cooled to 0-10° C. The mixture was quenched with water (0.67 L) and acidified to pH 2-3 with 6 N HCl (˜2.1 L). The mixture was extracted with ethyl acetate (2×2.33 L), washed with water (3.2 L), 10% brine (2.67 L) and dried over sodium sulfate (0.67 kg). The organic solvents are concentrated to a volume of ˜0.90 L to precipitate the product. Heptane (0.67 L) was added to further precipitate the product. The mixture was cooled and the solid was filtered and washed with heptane (2×0.33 L). The solid was dried (65° C., 10 mm Hg, 24-48 h) to give the product in 50% yield.

### EXAMPLE 5

### Ethyl 4-chloro-2,3-dihydro-3-hydroxy-7-methyl-2-oxo-1H-indolyl-3 acetate

A stirred mixture of ethyl acetate (0.725 kg) in THF (1.45 L) was cooled to −45±5° C. A 1 M THF solution of lithium bis(trimethylsilyl)amide (6.24 L) was added while maintaining the temperature between −45±5° C. After 30 min, a slurry of 4-chloro-7-methyl isatin (0.30 kg) in THF (1.50 L) was added to the solution and the mixture allowed to warm to room temperature over 30 mins. The reaction was complete when less than 5% of the isatin remains by TLC. The mixture was concentrated to a volume of 3.5 L and cooled to 0-10° C. The mixture was quenched with water (0.67 L) and acidified to pH 2-3 with 6 N HCl (˜2.1 L). The mixture was extracted with ethyl acetate (2×2.33 L), washed with water (3.2 L), 10% brine (2.67 L) and dried over sodium sulfate (0.67 kg). The organic solvents are concentrated to a volume of ˜0.90 L to precipitate the product. Heptane (0.67 L) was added to further precipitate the product. The mixture was cooled and the solid was filtered and washed with heptane (2×0.33 L). The solid was dried (65° C., 10 mm Hg, 24-48 h) to give the product in 50% yield.

The examples are provided for illustrative purposes and should not be construed as limiting the scope of the present invention.

1. A process of synthesizing a compound of formula (I): 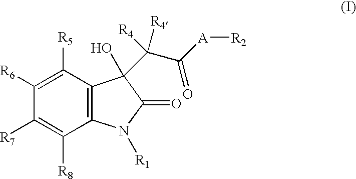
comprising the step of reacting a compound of formula (II) 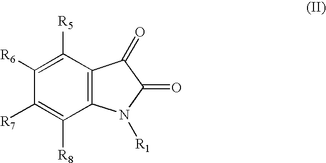
with M^{+-C(R}₄R_{4′})C(O)-A-R₂, wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms, all of which can be optionally substituted;
R₂ is a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all which may be optionally substituted or unsubstituted;
R₄ and R_{4′} are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an aryl of 6 to 12 carbon atoms, furanylmethyl, arylalkyl or alkylaryl of 7 to 12 carbon atoms, alkynyl of 2 to 7 carbon atoms, all of which can be optionally substituted, or R₄ and R₄ taken together with the ring carbon atom to which they are attached are a carbonyl group;
R₅-R₈ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or an alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, F, Cl, Br, I, CN, CF₃, NO₂, an alkylsulfinyl of 1 to 8 carbon atoms, an alkylsulfonyl of 1 to 6 carbon atoms, a pyrrolidinyl, or a thiazolidinyl, all of which can be optionally substituted;
R₁₂-R₁₃ are independently H, straight chain alkyl of 1 to 8 carbon atoms, branched alkyl of 3 to 12 carbon atoms, cycloalkyl of 3 to 12 carbon atoms, an aryl of 6 to 12 carbon atoms, or a heterocycloalkyl of 2 to 9 carbon atoms, all of which can be optionally substituted;
A is O or S; and
M⁺ is a metal cation.

2. The process of claim 1, further comprising the step of forming the compound of formula (II): 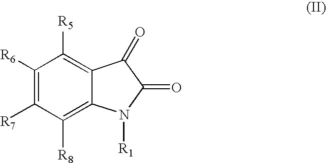
by cyclizing a compound of formula (VIII): 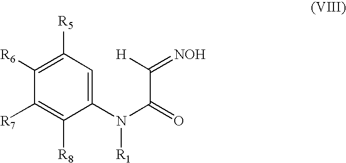
in the presence of an acid.

3. The process of claim 2, further comprising the step of reacting a compound of formula (VII): 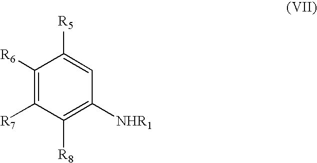
with a trihaloacetaldehyde hydrate and hydroxylamine hydrochloride to produce a compound of formula (VIII):

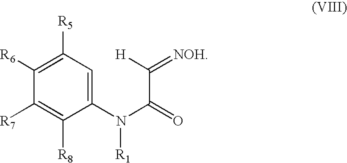

4. The process of claim 2, wherein the acid used to cyclize the compound of formula (VIII) is a strong mineral acid or a Lewis acid.

5. The process of claim 4, wherein the acid used to cyclize the compound of formula (VIII) is sulfuric acid.

6. The process of claim 3, wherein the trihaloacetaldehyde hydrate is chloral hydrate.

7. The process of claim 3, further comprising the proviso that no chromatographic purifications are performed to produce the compound of formula (I).

8. The process of claim 3, wherein the compounds used or formed are defined by:
R₁ is H or C₁-C₄ alkyl;
R₂ is a group selected from C₁-C₈ alkyl, C₇-C₁₂ alkylaryl, C₆-C₁₂ aryl and C₂-C₉ heterocycloalkyl;
R₄ and R_{4′}, are H;
R₅-R₈ are independently H, C₁-C₄ alkyl, F, Cl, Br, CN or CF₃; and
A is O or S.

9. The process of claim 8, wherein the compounds used or formed are defined by:
R₂ is C₁-C₄ alkyl or C₆-C₁₂ aryl;
A is O; and
M⁺ is Li.

10. The process of claim 9, wherein the compounds used or formed are defined by:
R₁ is H;
R₂ is t-butyl;
R₄ and R₄ are H;
R₅ is Br;
R₆ and R₇ are H; and
R₈ is CH₃.

11. A process of synthesizing a compound of formula (VI): 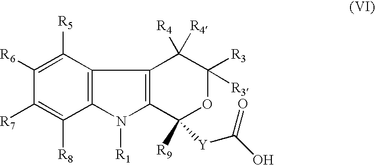
from a compound of formula (V) 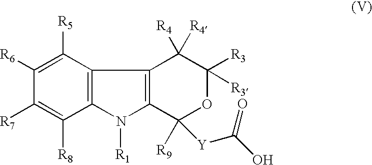
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms, all of which can be optionally substituted;
R₃ and R₃′ are H;
R₄ and R₄ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an aryl of 6 to 12 carbon atoms, a furanylmethyl, an arylalkyl or alkylaryl of 7 to 12 carbon atoms, an alkynyl of 2 to 7 carbon atoms, all of which can be optionally substituted, or R₄ and R₄ taken together with the ring carbon atom to which they are attached are a carbonyl group;
R₅-R₈ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or an alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, F, Cl, Br, I, CN, CF₃, NO₂, an alkylsulfinyl of 1 to 8 carbon atoms, an alkylsulfonyl of 1 to 6 carbon atoms, a pyrrolidinyl, or a thiazolidinyl, all of which may be optionally substituted;
R₁₂-R₁₃ are independently H, a straight chain alkyl of 1 to 8 carbon atoms, a branched alkyl of 3 to 12 carbon atoms, a cycloalkyl of 3 to 12 carbon atoms, an aryl of 6 to 12 carbon atoms, or a heterocycloalkyl of 2 to 9 carbon atoms, all of which may be optionally substituted;
R₉ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all of which can be optionally substituted; and
Y is a bond, CH₂, CH₂CH₂, aryl of 6 to 12 carbon atoms, or R₉ and Y together with the ring carbon atom to which they are attached may additionally form a spirocyclic cycloalkyl ring of 3 to 8 carbon atoms;
said process comprising the step of dissolving the compound of formula (V) in a solvent with a resolving agent to obtain the compound of formula (VI) by recrystallization.

12. The process of claim 11, further comprising the step of hydrolyzing a compound of formula (IV): 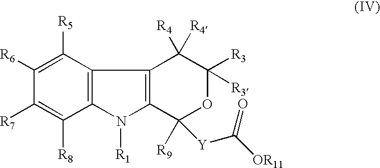
to produce the compound of formula (V):

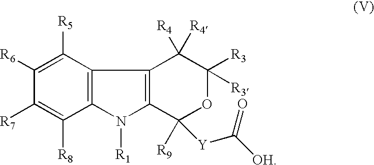

13. The process of claim 12, further comprising the step of reacting a compound of formula (III): 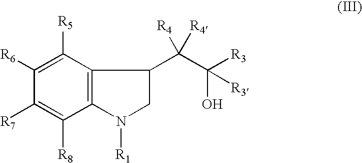
in the presence of an acid with a compound of formula R₉—C(O)—Y—COOR₁₁, wherein R₁₁ is alkyl, alkenyl, alkynyl, alkoxyalkyl, arylalkyl, alkylthioalkyl, cycloalkyl-alkyl, heterocycloalkyl, or aryl, all which may be optionally substituted or unsubstituted, to obtain the compound of formula (IV):

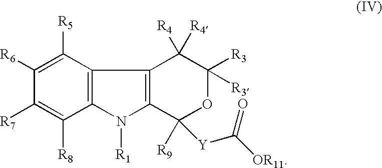

14. The process of claim 13, further comprising the step of reducing a compound of formula (I): 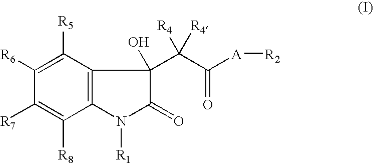
wherein R₂ is a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all which may be optionally substituted or unsubstituted; and
A is O or S;
to produce the compound of formula (III):

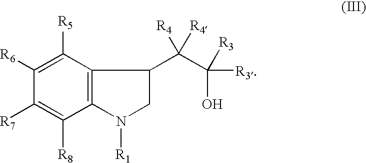

15. The process of claim 14, wherein the compounds used or formed are defined by:
R₁ is H or C₁-C₄ alkyl;
R₂ is a group selected from C₁-C₈ alkyl, C₇-C₁₂ alkylaryl, C₆-C₁₂ aryl and C₂-C₉ heterocycloalkyl;
R₃, R_{3′}, R₄ and R₄ are H;
R₅-R₈ are independently H, C₁-C₄ alkyl, F, Cl, Br, CN or CF₃;
A is O or S;
R₉ is H or C₁-C₈ alkyl; and
Y is a bond, CH₂, CH₂CH₂, or C₆-C₁₂ aryl, or R₉ and Y together with the ring carbon atom to which they are attached may additionally form a spirocyclic cycloalkyl ring of 3 to 8 carbon atoms.

16. The process of claim 11, wherein the resolving agent is selected from the group consisting of (+) cinchonine, (−) burcine, (−) ephedrine, R-(−)-2-amino-1-butanol, R-(−)-2-amino-1-propanol, R-(−)-2-amino-3-methyl-1-butanol, R-(+)-2-amino-3-3-dimethylbutane, R-(+)-2-amino-3-phenyl-1-propanol, (R)-phenylethylamine, (S)-phenylethylamine, S-(+)-2-amino-1-butanol, S-(+)-2-amino-1-propanol, S-(+)-2-amino-3-methyl-1-butanol, N-methyl-D-glucamine, (R)-(+)-N, N-dimethyl-1-phenethylamine, (S)-(−)-N,N-dimethyl-1-phenethylamine, (1R,2R)-(−)-pseudoephedrine, (1R,2S)-(−)-ephedrine, (1S,2S)-(+)-pseudoephedrine, (R)-(−)-ephinephrine, nicotine, quinine, and strychine.

17. The process of claim 16, wherein the resolving agent is (+) cinchonine.

18. The process of claim 13, wherein the acid used in converting the compound of formula (III) to the compound of formula (IV) is a Lewis acid.

19. The process of claim 18, wherein the Lewis acid is selected from the group consisting of BF₃.Et₂O, ZnCl₂, AlCl₃, BCl₃, BBr₃ and FeCl₃.

20. The process of claim 14, further comprising the step of reacting the compound of formula (I), wherein at least one of R₅-R₈ are a leaving group selected from the group consisting of halo, —O-triflate, —O-mesylate, or —O-tosylate, with a reagent to produce a compound where the leaving group has been replaced with a group selected from a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or an alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, CN, CF₃, NO₂, an alkylsulfinyl of 1 to 8 carbon atoms, an alkylsulfonyl of 1 to 6 carbon atoms, a pyrrolidinyl, and a thiazolidinyl, all of which can be optionally substituted.

21. The process of claim 12, further comprising the step of reacting the compound of formula (IV), wherein at least one of R₅-R₈ are a leaving group selected from the group consisting of halo, —O-triflate, —O-mesylate, or —O-tosylate, with a reagent to produce a compound where the leaving group has been replaced with a group selected from a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or an alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, CN, CF₃, NO₂, an alkylsulfinyl of 1 to 8 carbon atoms, an alkylsulfonyl of 1 to 6 carbon atoms, a pyrrolidinyl, and a thiazolidinyl, all of which can be optionally substituted.

22. The process of claim 14, further comprising the proviso that no chromatographic purifications are performed to produce the compound of formula (VI).

23. The process of claim 15, wherein the compounds used or formed are defined by:
R₁ is H or C₁-C₄ alkyl;
R₂ is C₁-C₄ alkyl or C₆-C₁₂ aryl;
R₅-R₈ are independently H, C₁-C₄ alkyl, F, Cl, Br, CN or CF₃;
A is O or S;
R₉ is H or C₁-C₄ alkyl; and
Y is CH₂.

24. The process of claim 23, wherein the compounds used or formed are defined by:
R₁ is H;
R₅-R₈ are independently H, a straight chain alkyl of 1 to 4 carbons, F, Cl, Br or CN;
R₉ is H or a straight chain alkyl of 1 to 4 carbons; and
A is O.

25. The process of claim 24, wherein the compounds used or formed are defined by:
R₂ is t-butyl;
R₅ is CN;
R₆ and R₇ are H;
R₈ is CH₃; and
R₉ is n-propyl.

26. A compound of formula (I): 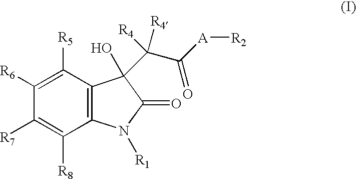
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms, all of which can be optionally substituted;
R₂ is a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl of 2 to 12 carbon atoms, an arylalkyl or alkylaryl of 7 to 12 carbon atoms, an alkylthioalkyl of 2 to 16 carbon atoms, a cycloalkyl-alkyl of 4 to 24 carbon atoms, an aryl of 6 to 12 carbon atoms, or a heterocycloalkyl of 2 to 9 carbon atoms, all which may be optionally substituted or unsubstituted;
R₄ and R_{4′} are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an aryl of 6 to 12 carbon atoms, a furanylmethyl, an arylalkyl or alkylaryl of 7 to 12 carbon atoms, an alkynyl of 2 to 7 carbon atoms, all of which can be optionally substituted, or R₄ and R_{4′} taken together with the ring carbon atom to which they are attached are a carbonyl group;
R₅-R₈ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, an aryl of 6 to 12 carbon atoms, a heterocycloalkyl of 2 to 9 carbon atoms, a furanylmethyl, an arylalkyl or alkylaryl of 7 to 12 carbon atoms, an alkynyl of 2 to 7 carbon atoms, a phenylalkynyl, an alkoxy of 1 to 8 carbon atoms, an arylalkoxy of 7 to 12 carbon atoms, a fluoroalkoxy of 1 to 12 carbon atoms, an alkylthio of 1 to 6 carbon atoms, a trifluoromethoxy, a trifluoroethoxy, a trifluoromethylthio, a trifluoroethylthio, an acyl of 1 to 7 carbon atoms, COOH, COO-alkyl, CONR₁₂R₁₃, F, Cl, Br, I, CN, CF₃, NO₂, an alkylsulfinyl of 1 to 8 carbon atoms, an alkylsulfonyl of 1 to 6 carbon atoms, a pyrrolidinyl, or a thiazolidinyl, all of which can be optionally substituted;
R₁₂-R₁₃ are independently H, a straight chain alkyl of 1 to 8 carbon atoms, a branched alkyl of 3 to 12 carbon atoms, a cycloalkyl of 3 to 12 carbon atoms, an aryl of 6 to 12 carbon atoms or heterocycloalkyl of 2 to 9 carbon atoms, all of which can be optionally substituted; and
A is O or S.

27. The compound of claim 26, wherein:
R₁ is H or C₁-C₄ alkyl;
R₂ is a group selected from C₁-C₈ alkyl, C₇-C₁₂ alkyl-aryl, C₆-C₁₂ aryl and C₂-C₉ heterocycloalkyl;
R₄ and R_{4′} are H;
R₅-R₈ are independently H, C₁-C₄ alkyl, F, Cl, Br, CN or CF₃; and
A is O.

28. The compound of claim 27, wherein R₂ is a group selected from C₁-C₄ alkyl or aryl and A is O.

29. The compound of claim 28, wherein:
R₁ is H;
R₂ is t-butyl;
R₄ and R₄ are H;
R₅ is Br;
R₆ and R₇ are H; and
R₈ is CH₃.
